(19) United States
(12) Patent Application Publication
(10) Pub. No.: US 2025/0367464 A1
(43) Pub. Date: Dec. 4, 2025

(54) DEVICE TO STIMULATE THE CENTRAL NERVOUS SYSTEM OF THE BODY USING ELECTROMAGNETIC WAVES TO REDUCE THE EFFECTS OF MULTIPLE SCLEROSIS AND OPTIMIZE THE TRANSMISSION OF NERVE MESSAGES IN THE AXON OF THE NERVE CELL

(71) Applicant: Vesal Abbasian, Tehran (IR)

(72) Inventor: Vesal Abbasian, Tehran (IR)

(21) Appl. No.: 18/676,569

(22) Filed: May 29, 2024

(51) Int. Cl. A61N 2/00; A61G 5/12; A61N 2/02

(52) U.S. Cl. CPC A61N 2/006; A61G 5/121; A61N 2/02

(57) ABSTRACT

In the invention of a device to stimulate the central nervous system of the body using electromagnetic waves to reduce the effects of multiple sclerosis and optimize the transmission of nerve messages in the axon of the nerve cell, a wheelchair is presented by using an electromagnetic wave generator and wires, the magnetic field was transferred along the spine and back of the patient's neck and head to stimulate the disabled nerves and it controls this process using artificial intelligence. MS is an autoimmune disease in which the immune system unknowingly attacks a person's nervous system and reduces or interrupts signal transmission from the nervous system. The above invention is induced to the required areas with the help of electromagnetic waves and its parameters can be controlled with the help of artificial intelligence. This system can be used in different environments including hospitals, clinics and even personalized for each person at home.

### TECHNICAL FIELD OF THE INVENTION

The present invention is equipped with electronic equipment and communication modules and advanced devices that are provided for the treatment of MS disease. This system stimulates nerve cells and controls this process using artificial intelligence using new technologies such as artificial intelligence and electromagnetic waves.

### PRIOR ARTS

The researches show that MS is an autoimmune disease which has destructive effects on the neural system. In this disease, the body's immune system mistakenly attacks nerve cells in the brain and spinal cord. These attacks lead to destruction and changes caused by inflammation and destruction of different areas of nerve cells, which can lead to various symptoms.

The mechanism of destructing the nerve cells in the MS extensively has been studied; however, it generally contains multiple steps. The first step is the stimulation of the body's immune system to nerve cells by autoantibodies that are incorrectly produced. Then, these autoantibodies and other immune cells somehow attack nerve cells, causing inflammation and damage around them. This inflammation and cell damage over time leads to the destruction and poor function of nerve cells, which results in the symptoms of MS.

As a result, damage to nerve cells in MS not only causes physical destruction in nerve cells but also causes inflammation and chemical changes in the environment around the cells, both of which lead to various symptoms in people with MS. Therefore, various designs and inventions have been made in this field, which can be mentioned below.

A Japanese invention with patent No. JP6735320B2 which is granted dated May 8, 2020 titled “Treatment device for multiple sclerosis, deep vein thrombosis, and/or pulmonary embolism” relates to a treatment device for MS, deep vein thrombosis and/or pulmonary embolism. This device includes a power source, an electrode and a control unit. The power source produces electromagnetic waves with a specific frequency and amplitude. These waves can be of different types such as radio waves, microwaves or infrared waves. The electrode applies electromagnetic waves to the patient's body. Electrodes can be placed on the patient's skin or directly implanted in the patient's body. The control unit controls the frequency and amplitude of electromagnetic waves. The control unit can also control the time and duration of the treatment. The function of this treatment device is that the power source produces electromagnetic waves with a specific frequency and amplitude, and the electrode applies electromagnetic waves to the patient's body. Electromagnetic waves penetrate body tissues and cause heat. Heat increases blood circulation and helps oxygenate the tissues. Increasing blood circulation and oxygen supply to tissues helps to reduce inflammation and pain. This device is effective for treating MS, deep vein thrombosis and/or pulmonary embolism. This device can significantly reduce the symptoms of these diseases and improve the quality of life of patients. The frequency and amplitude of the electromagnetic waves produced by the energy source depend on the type of disease being treated. For example, to treat MS, the frequency of electromagnetic waves is usually between 100 MHz and 1 GHz and the amplitude is between 1 and 10 watts. Electrodes can be placed on the patient's skin or directly implanted in the patient's body. Electrodes on the skin are usually made of silicone or a conductive metal such as copper or silver. Implanted electrodes are usually made of titanium or platinum. The location of the electrodes depends on the type of disease being treated. For example, to treat MS, electrodes are usually placed on the head, spine, and limbs. The control unit controls the frequency and amplitude of the electromagnetic waves as well as the time and duration of the treatment. The control unit can also record information related to the treatment, such as the frequency and amplitude of the applied electromagnetic waves, the treatment time, and the patient's body temperature.

An US invention with patent No. U.S. Pat. No. 9,005,102B2 which is granted dated 14 Apr. 2015 titled “Method and apparatus for electrical stimulation therapy” relates to energy emitting systems which include an adjustable conductive coil configured to generate a magnetic or electromagnetic field focused on a target nerve. The adjustable or movable nature of the coil allows the conductive coil to conform to, accommodate, or be positioned on a particular anatomical structure of a patient to position the coil in proximity to the underlying target nerve. In certain embodiments, methods of magnetic induction therapy are provided which include positioning a conductive coil relative to a portion of a patient's body by adjusting the central aperture of the coil such that the coil may conform to, accommodate or be positioned on the portion of the patient's body in proximity to the underlying target nerve. This device includes an electric current generator, a control unit and electrodes. The generator produces an electric current with a certain frequency and amplitude, and the control unit controls the frequency and amplitude of the electric current, as well as the time and duration of the treatment, the electrodes apply the electric current to the patient's body. Electrodes can be of different materials such as silicon, conductive metal or conductive gel. The location of the electrodes depends on the type of disease being treated. This method and device is used to treat pain caused by various diseases such as arthritis, chronic back pain, fibromyalgia, sports injuries and neuropathy. The exact mechanism of action of this device is not yet fully understood, but it is believed that the electric current produced by the device produces effects such as pain relief, reducing inflammation, and improving blood circulation in the body. This therapy device can use different types of electrical stimulation to treat pain, including transdermal electrical stimulation, functional muscle electrical stimulation, and spinal cord electrical stimulation. The operation of this method and device is as follows:

First, the patient lies on the treatment bed. Electrodes are placed on the patient's skin at designated locations. The control unit is turned on and the frequency, amplitude and duration of the treatment are set. The generator produces electric current with the set frequency and amplitude. Electric current is applied to the patient's body through electrodes. The patient may feel tingling, tingling or mild pain during the treatment. After the treatment time is over, the device will turn off automatically. This treatment device can use different types of electrical stimulation to treat pain, including:

Transcutaneous electrical stimulation: TENS is a type of electrical stimulation that is applied through electrodes placed on the skin and is commonly used to treat acute and chronic pain.

Functional Electrical Muscle Stimulation: FES is a type of electrical stimulation used to stimulate muscles and is commonly used to treat muscle weakness caused by stroke, spinal cord injury, and other conditions.

SCS is a type of electrical stimulation that is applied to the spinal cord. It is usually used to treat severe chronic pain that does not respond to other treatments.

Another US invention with patent No. U.S. Pat. No. 11,826,579B2 which is granted dated 28 Nov. 2023 titled “System and method for applying a low frequency magnetic field to biological tissues” is a system and method for applying a low strength, low frequency magnetic field therapy to biological tissues. A coil is excited with a low frequency oscillating current, e.g., 10-1000 Hz. The coil is, e.g., 5-200 turns, having a diameter of 2-20 mm, and produces a magnetic field strength of about 0.01-5 mTelsa at a distance of 1 cm from the coil, or a cover over the coil, into the tissue. The current is preferably controlled by a smartphone or other programmable device controlled by a downloadable app by a PEMF program which may be separately downloaded or updated, and may be provided through an audio jack. Alternately, a digital interface and/or wireless interface may control the current. An app on the smartphone may be used to control the frequency, amplitude/envelope modulation, waveform, duration, etc. of the oscillation. The coil may be in mineral housing with a simple filter, and TRRS-type audio jack. The operation of this system is as follows, the patient lies on the treatment bed. The conductive coil is placed on the treated area. The control unit is turned on and the frequency, amplitude and duration of the treatment are set. The current source applies an electric current to the conductive coil. The conductive coil produces a low-frequency magnetic field that is applied to biological tissues. The patient may feel tingling, prickling or mild pain during the treatment. After the treatment time is over, the device will turn off automatically. The low-frequency magnetic field produced by the system is thought to help with effects such as pain relief from various ailments. It does this by stimulating the release of endorphins, which are natural chemicals in the body that block the sensation of pain. A low frequency magnetic field can help reduce inflammation in the body. Inflammation is a key factor in many diseases, including pain. A low-frequency magnetic field can help improve blood circulation in the treated area. This can help deliver more oxygen to the tissues and remove waste materials.

Also an US invention with publication No. US20170312536A1 which is filed dated 13 Apr. 2017 titled “Devices and methods of low frequency magnetic stimulation therapy” are devices and methods for modulating the electrical activity of a brain in a targeted manner using a weak magnetic field, generally field with a strength of less than about 100 Gauss. The magnetic field is varied over time in a periodic manner, generally with a frequency tuned to specifically affect one of the intrinsic frequencies of the brain, optionally the alpha frequency. The “Low Field Magnetic Stimulation” devices and methods described herein modulate the electrical activity of a brain without requiring medication. Methods and devices described herein gently “tune” the brain and affect mood, focus, and cognition of human subjects. This invention includes two main elements: a coil that produces the magnetic field necessary to stimulate the brain. The coil can be made of different materials such as copper, silver or aluminum. The number of turns of the coil and its diameter can affect the intensity of the magnetic field. A current source also applies electric current to the coil. The frequency and amplitude of the electric current applied to the coil can affect the frequency and amplitude of the magnetic field.

An US invention with patent No. U.S. Pat. No. 10,703,811B2 which is granted dated Jul. 7, 2020 titled “Methods and compositions for treating multiple sclerosis” relates to the compositions and methods include agents that inhibit inflammasome signaling in the mammal such as antibodies directed against inflammasome components used alone or in combination with extracellular vesicle uptake inhibitor(s). The items described in this invention include the following: Antibodies that are introduced into the body against inflammasome components. These antibodies are specific molecules that bind to the components of the inflammasome and deactivate them. Also, inhibitors of the uptake of extracellular vesicles, which are extracellular vesicles that are small sacs that cells use to communicate with each other. This invention also proposes inhibitors that prevent the uptake of these vesicles and thereby affect inflammasome signaling. The invention mentions that these compounds can be used alone or in combination with extracellular vesicle uptake inhibitors for the treatment of MS. These methods and compounds have several advantages over current treatments for MS, such as drugs. These methods and compounds specifically target the inflammasome, which can help reduce their side effects. Studies have shown that these methods and compounds can significantly improve MS symptoms. These methods and compounds are generally considered safe and secure.

Also an US invention with patent No. U.S. Pat. No. 9,757,584B2 which is granted dated Dec. 9, 2017 titled “Methods and devices for performing electrical stimulation to treat various conditions” is a systems and/or methods for electromagnetic induction therapy are provided. One or more ergonomic or body contoured applicators may be included. The applicators include one or more conductive coils configured to generate an electromagnetic or magnetic field focused on a target nerve, muscle or other body tissues positioned in proximity to the coil. One or more sensors may be utilized to detect stimulation and to provide feedback about the efficacy of the applied electromagnetic induction therapy. A controller may be adjustable to vary a current through a coil to adjust the magnetic field focused upon the target nerve, muscle or other body tissues based on the feedback provide by a sensor or by a patient. In certain systems or methods, pulsed magnetic fields may be intermittently applied to a target nerve, muscle or tissue without causing habituation.

An US invention with patent No. U.S. Pat. No. 9,278,231B2 which is granted dated Aug. 3, 2016 titled “Sequentially programmed magnetic field therapeutic system (SPMF)” is a comprehensive system for inducing cellular regeneration and/or degeneration processes and methods of treatment based on such processes through generating and applying a sequentially programmed magnetic field (SPMF) to the area to be treated. In the case of regeneration and degeneration of cells, the pulsing frequencies are in the range of about 0.1 to about 2000 Hz based on the indication of the disease type which was determined by either the patient's MRI, CT, Ultrasound or other diagnostic information. Methods for treating diseases or conditions that will benefit from regeneration and/or degeneration of cells. For example, methods for treating cancer, arthritis, neuro degeneration conditions, such as the age-related progressive loss of nerve cells, Alzheimer's, Parkinson's, ALS, and Huntington's disease, retinal degeneration, and other damage to sensory systems (e.g., visual, auditory, somatosensory), in stroke, head and spinal trauma, epilepsy, in drug and alcohol abuse, in infectious diseases, in exposure to industrial and environmental toxicants, and, perhaps, in mental disorders and chronic pain.

A Spanish invention with patent No. ES2906959T3 which is granted dated 21 Apr. 2022 titled “Magnetic stimulation devices for therapeutic treatments” is a stimulation device for aesthetic treatment that uses a time-varying magnetic field and a radiofrequency field to provide muscle contraction and heating of at least one biological structure, wherein the stimulation device comprises:—at least one first device switching (11),—at least one first energy storage device (13),—at least one first magnetic field generating device (12), and—at least one first radiofrequency electrode, in which the first device switching (11) is configured to discharge the first energy storage device (13) to provide an electrical current flow to the first magnetic field generating device (12), the first magnetic field generating device being configured to generate biphasic pulses of a time-varying magnetic field, being used for magnetic treatment, with a mag flux density in a range of 0.2 to 7 Tesla, a pulse duration in a range of 10 to 1000 μs and a repetition rate of magnetic pulses in a range of 1 to 700 Hz, in which each pulse comprises a pulse of the magnetic field time variable; wherein the time-varying magnetic field generated by the first magnetic field generating device is capable of causing muscle contraction; wherein the stimulation device further comprises an HF frequency generator to provide energy for the radiofrequency treatment; wherein the at least one first radiofrequency electrode is configured to generate a radiofrequency field for heating at least one target biological structure to a temperature in a range of 37° C. to 60° C.

Another US invention with patent No. U.S. Pat. No. 10,556,121B2 which is granted dated Nov. 2, 2020 titled “Method and apparatus for electromagnetic treatment of multiple sclerosis” is a methods and apparatuses (including systems, devices, etc.) for the treatment of neurological disorders, and particularly multiple sclerosis (MS) by the application of electromagnetic fields (EMF), and in particular, pulsed electromagnetic fields (PEMF), including a subset of PEMF in a radio frequency domain (e.g., pulse-modulated radio frequency or PRF). This treatment may prevent or delay the onset, and/or may reduce the severity of the onset of MS and MS-related symptoms. A torso garment configured to be worn on a subject's torso, the torso garment having a back and a neck portion, the back including a middle region. A plurality of wire coil PEMF applicators extending down the middle region of the back to treat the subject's spine, wherein the plurality of wire coil PEMF applicators comprises a plurality of substantially coplanar loops arranged adjacent to each other in a line down the back. a neck wire coil PEMF applicator on the neck portion to treat the patient's brain stem and cervical spine; and a PEMF signal generator integrated into the torso garment, wherein the PEMF signal generator is configured to deliver a treatment dose comprising a PEMF signal, wherein the PEMG signal generator is connected to the plurality of wire coil PEMF applicators and the neck wire coil PEMF applicator.

And also an US invention with patent No. U.S. Pat. No. 10,806,942B2 which is granted dated 20 Oct. 2020 titled “System and method for applying a low frequency magnetic field to biological tissues” a system and method for applying a low strength, low frequency magnetic field therapy to biological tissues. A coil is excited with a low frequency oscillating current, e.g., 10-1000 Hz. The coil is, e.g., 5-200 turns, having a diameter of 2-20 mm, and produces a magnetic field strength of about 0.01-5 mTelsa at a distance of 1 cm from the coil, or a cover over the coil, into the tissue. The current is preferably controlled by a smartphone or other programmable device controlled by a downloadable app in accordance with a PEMF program which may be separately downloaded or updated, and may be provided through an audio jack. Alternatively, a digital interface and/or wireless interface may control the current. An app on the smartphone may be used to control the frequency, amplitude/envelope modulation, waveform, duration, etc. of the oscillation. The coil may be in mineral housing with a simple filter, and TRRS-type audio jack. This system consists of a coil, a current source and a controller. The coil is connected to the current source. The controller adjusts the frequency, intensity and duration of the magnetic field. The electric current passes through the coil and the field is magnetically produced. A magnetic field is applied to biological tissues.

Today, a wide range of diseases can be treated or controlled using waves and according to research, PEMF consists of higher frequency (ie, carrier frequency) sine wave pulses with a carrier frequency of 10 MHz, with pulses repeated between 0.01 and 10 Hz and field strengths between 1 and 200 mGauss, are significantly more effective (more than 20% increase) than other parameters and methods. The presented ranges for these parameters, especially for carrier frequency, pulse duration and pulse rate, show a significant improvement over stimulation outside these ranges.

Also, in order to reduce the harmful effects on the body, according to research, the amount of radiation on the body must be followed according to the protocols and standards proposed by the relevant organizations, one of which is SAR. SAR is the abbreviation of “Specific Absorption Rate” means. SAR measures the amount of radio frequency (RF) energy absorbed by the human body and is measured in watts per kilogram (W/kg). According to the limits announced by the International Commission on Non-Ionizing Radiation Protection (ICNIRP) and the World Health Organization (WHO), the maximum SAR allowed for the head and trunk is 20 watts/kg (W/kg) and for the extremities such as hands and ankles has determined 40 watts/kg (W/kg) in an average time of 6 minutes. Considering the mentioned protocols, the present invention has presented a smart wheelchair for the treatment or control the MS.

### DESCRIPTION OF THE INVENTION

The present invention provided a smart wheelchair, which includes a neck support (FIG. 3, No. 101) that is designed according to the body ergonomics for the patient's comfort, and has the ability to adjust its angle with the seat support.

A signal generator (FIG. 6, No. 205), several coils (FIG. 3, No. 103), a control unit (FIG. 5), a weight control unit (FIG. 3, No. 104) and a power supply unit (FIG. 6, No. 203) including a rechargeable battery to power the system and a wireless unit (FIG. 6, No. 201) to send and receive information to the PC and an announcement unit (FIG. 6, No. 204) is a mobile phone software with artificial intelligence algorithms.

The signal generator can from different types of such as direct current generator, alternating current generator or pulsed electromagnetic signal generator that in this invention, a pulsed electromagnetic signal generator has been used that has been converted into a pulsed magnetic field by a coil. The magnetic field is a variable pulse that alternately turns on and off. The frequency, pulse duration, pulse speed, field strength, field direction by the generator depends on the type of disease being treated. In this invention, in order to treat MS disease, the parameters of the signal produced by the signal generator have been considered in the form of sinusoidal pulses with a carrier frequency of 10 MHz, which pulses are repeated between 0.5 and 5 Hz and have a field strength between 1 and 200 milligauss. The duration of the treatment is defined as once a day with duration of 12 minutes or multiple time intervals during the day with a shorter amount of time.

The electromagnetic fields produced by the signal generator can stimulate the nerves through a process called electromagnetic induction. This phenomenon is based on the electromagnetic induction of Faraday's law. According to Faraday's law of electromagnetic induction, a change in magnetic field can induce an electric current in a coil or conductor. The mechanism of this process is described below:

The pulsed electromagnetic signal produced by the signal generator is transmitted to the coils through the embedded cables (FIG. 3, No. 102). The conductive coil can be made of different materials such as copper, silver or aluminum. The number of turns of the coil and its diameter can affect the intensity of the magnetic field produced. The conductive coil can be applied individually or as a coil array. The location of the conductive coil is in line with the location of the patient's spine, back of the neck and head.

When electromagnetic signals pass through the coils, it creates a magnetic field around the coil. By changing in the magnetic field (any change in intensity, direction, or both) when a magnetic field changes rapidly near a nerve, it induces a weak electric field in the conductive tissue surrounding the nerve. This conductive tissue includes liquids and the body electrolytes.

This induced magnetic field makes a weak electric current in the nerve tissue. This current moves according to the direction of the electric field and can affect the electrical activity of nerve cells and stimulate neurons.

Depending on the intensity, direction, and duration of the electrical current, stimulation can cause depolarization (Depolarization is the process in which the electrical polarity of the cell membrane is temporarily reversed). In nerve and muscle cells, the resting cell membrane has a negative charge on the inside and a positive charge on the outside. This electrical potential difference is called membrane potential. In depolarization, ion channels in the cell membrane open and allow sodium ions (Na+) to flow into the cell. This makes the inside of the cell more positive and the outside of the cell more negative. This temporary change in electrical polarity is called an action potential and is essential for the transmission of electrical signals in the nervous system and muscle contraction. This change in electrical activity can eventually cause nerve impulses.

The key point is that this magnetic field is changing and indirectly creating a stimulating electric current in the nerve tissue. The electromagnetic field itself does not directly interact with the nerves.

In order to prevent the patient from getting used to the treatment process, one of the treatment parameters in the control unit is changed every time the signal is applied. The control unit used a micro which this micro controls the frequency, duration of the pulse, pulse speed, field strength and field direction as well as duration of treatment according to the patient's condition. The control unit can also record information related to the treatment, such as the frequency and amplitude of the applied electric current, the treatment time and the patient's pain level and display information on the computer with the help of a smartphone application or through a computer user interface. Also, this unit transmits the information obtained from the patient's condition to the PC for processing with the help of the wireless system, which gives the device the ability to make decisions through the internet and with the help of artificial intelligence. in this way, it first receives the information related to the patient's condition and with the help of the existing database, it makes the necessary decisions for the changes of each of the effective parameters such as frequency, pulse duration, pulse speed, field strength, field direction, or treatment time changes and sends it through the wireless system to the control unit installed on the wheelchair.

The patient's information such as age, sex, severity of the disease and the extent of the damaged nerve path and any other information that has an impact on the treatment process are sent to the smart wheelchair control unit through a PC or smart phone application via a wireless network. By using this information, the micro activates only the coil for that treatment range and adjusts the parameters of the electromagnetic signal.

The power supply unit is installed to feed the circuits of this system, which uses a rechargeable lithium ion battery, one of the batteries is placed in the circuit and the other is placed as a supporter in the charging circuit. As soon as the main battery is discharged, the supporter battery enters the circuit and the first battery is charged.

A weight control unit is installed on the seat of this wheelchair, which uses four load cells in the four corners of the seat. These load cells have two uses. The first use is to detect the presence of a person on the chair that in this case, the system starts working and if the measured weight becomes zero, it sends the system shutdown message to the control unit. The second application, based on the information received from these load cells, the measured weight of the two rear load cells together and the two front load cells together are checked in the control unit so that the weight is evenly distributed on the right and left side of the seat and if there is a conflict, the control unit recognizes that the person is not in a proper position and sends a signal to the mobile phone or computer screen so that the person can correct the way of sitting and the patient is informed through the speakers embedded in the chair. This action aims to align the patient's body with the path of the coils so that the coils are placed exactly in line with the patient's spine.

The communication unit can be a relatively short-range communication module. In the communication unit, based on the conditions and needs, one of the methods of Wi-Fi or Bluetooth may be used, which can communicate and send and receive data.

The application embedded in the present invention is designed with several goals. The first goal is to make the system user friendly to communicate with the operator or the patient so that the patient can be aware the treatment process during the treatment. Also, depending on the patient's condition, such as weight, sex, age, etc., the amount of each treatment parameter will be different. By installing this application before starting the treatment, the user enters this information so that the specific treatment program is uploaded to the control unit through artificial intelligence and then it starts working.

The function of this invention is that the patient sits on the wheelchair and puts the head on the embedded support according to the ergonomics of the body. The coils are placed on the spine and head and neck. The electromagnetic signal generation source is turned on and the frequency, pulse duration, pulse speed, field strength and field direction are adjusted with the help of the control unit. The coil induces the generated magnetic field to the body based on the degree of damage of each area and stimulates its electrical activity. The patient may feel tingling, prickling or mild pain during the treatment. After the end of the treatment time, the device automatically goes into standby mode and after the patient gets up from the chair, the load cells on the chair send the shutdown signal to the control unit to turn off the system.

As mentioned multiple sclerosis (MS) attacks the nervous system and neurons, causing the loss of the fatty layer of myelin on the axon part of the nerve cell. This layer is responsible for nerve signal transmission. With the damage and destruction of this layer or the inflammation caused by the destruction of this fat layer, electrical signals are either not transmitted or have a low transmission speed. The mechanism of effect of magnetic stimulation is that it can increase the activity of neurons in the brain in such a way as to repair the damaged tissue to transmit the electrical signal.

This invention accelerates the binding of intracellular ions, strengthens biochemical signaling pathways and in addition, by releasing growth factors and cytokines and thereby regulating the expression of specific genes, it will be successful in reducing the symptoms associated with MS disease. The invention will also lead to the use of electromagnetic waveforms to stimulate the production of cytokines and growth factors in living cells, offering potential benefits in the management of MS symptoms.

### BRIEF DESCRIPTION OF THE FIGURES

FIG. 1 shows 3 views of the smart wheelchair.

FIG. 2 shows sending and receiving information between a smart wheelchair with a computer and a smart mobile phone wirelessly.

FIG. 3 shows the isometric view of the smart wheelchair, which includes:
101: Neck support with a coil to induce electromagnetic waves to the nerve path of the neck
102: The connecting wire between the signal generator and the coil
103: Coil for induction of electromagnetic waves to the spinal nerve path
104: Location of load cells on the wheelchair seat
105: Control unit

FIG. 4 shows the exploded view of the smart wheelchair.

FIG. 5 shows the overview of the control unit.

FIG. 6 shows the internal view of the control unit, which includes:
201: Wireless sending and receiving module
202: Processor unit
203: Power supply unit
204: Speaker

FIG. 7 shows the exploded view of the control unit.

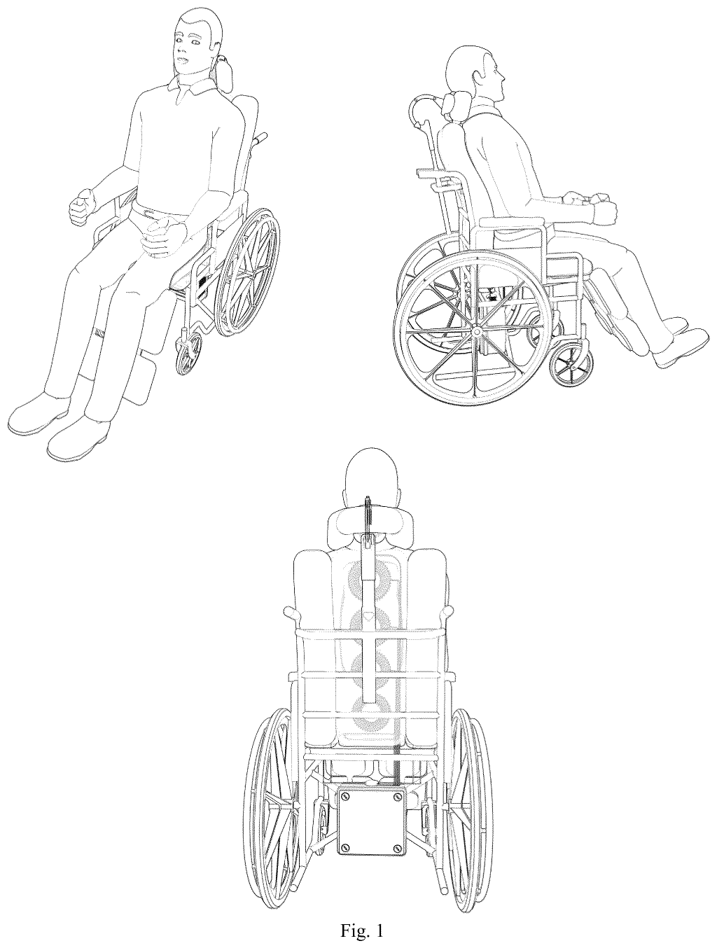

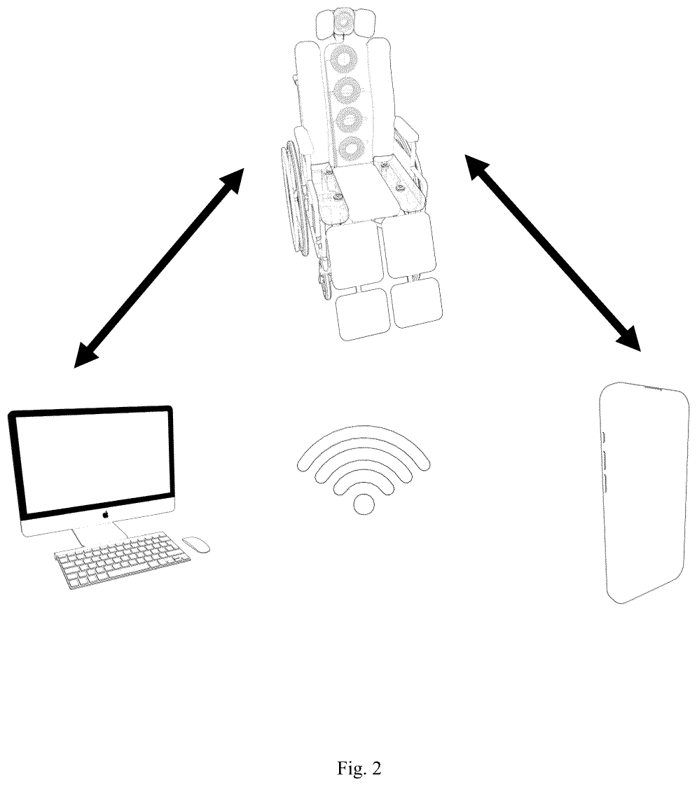

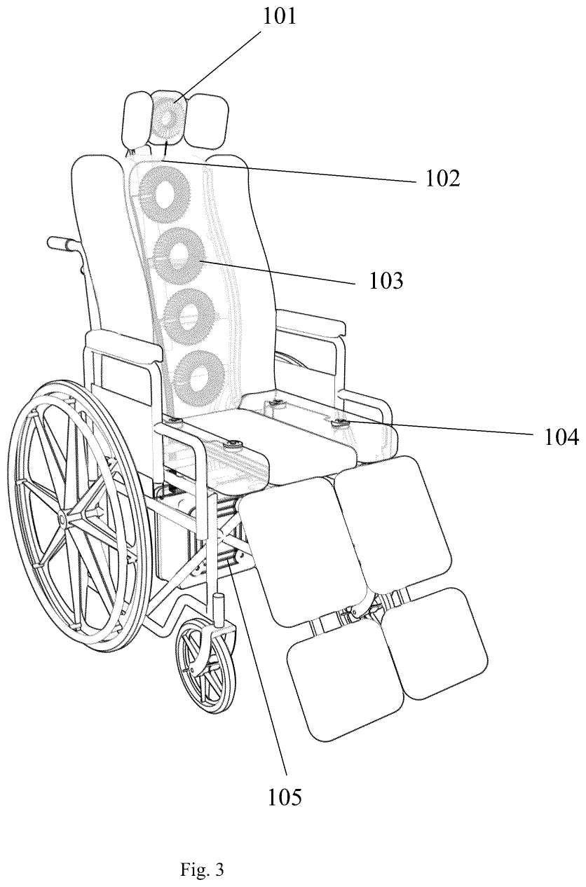

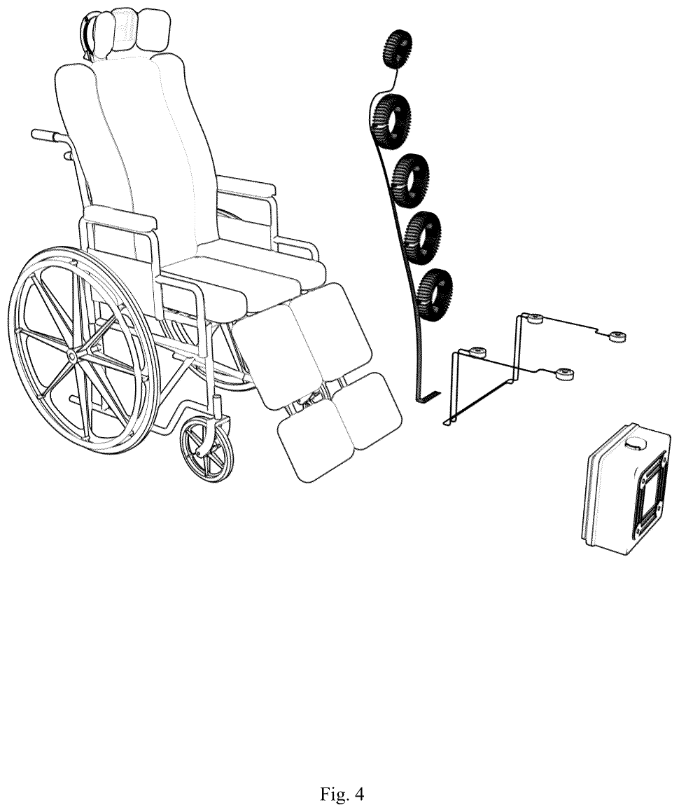

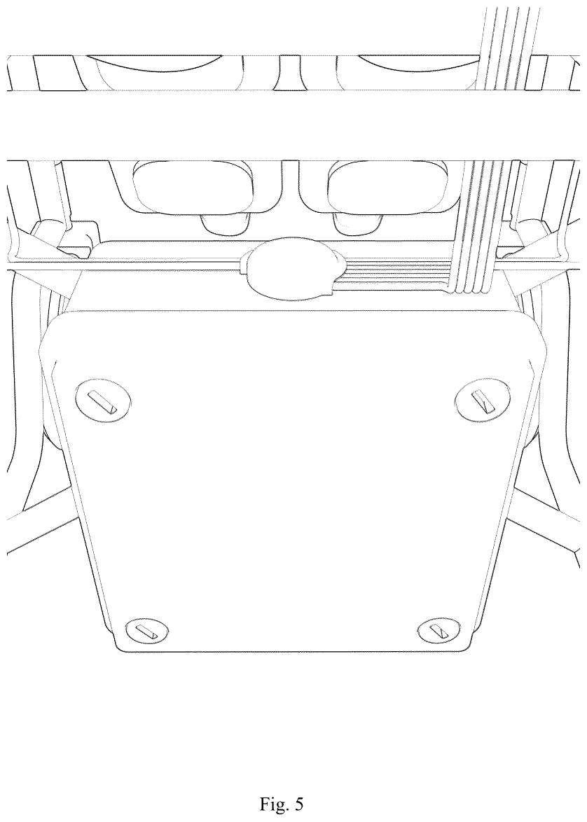

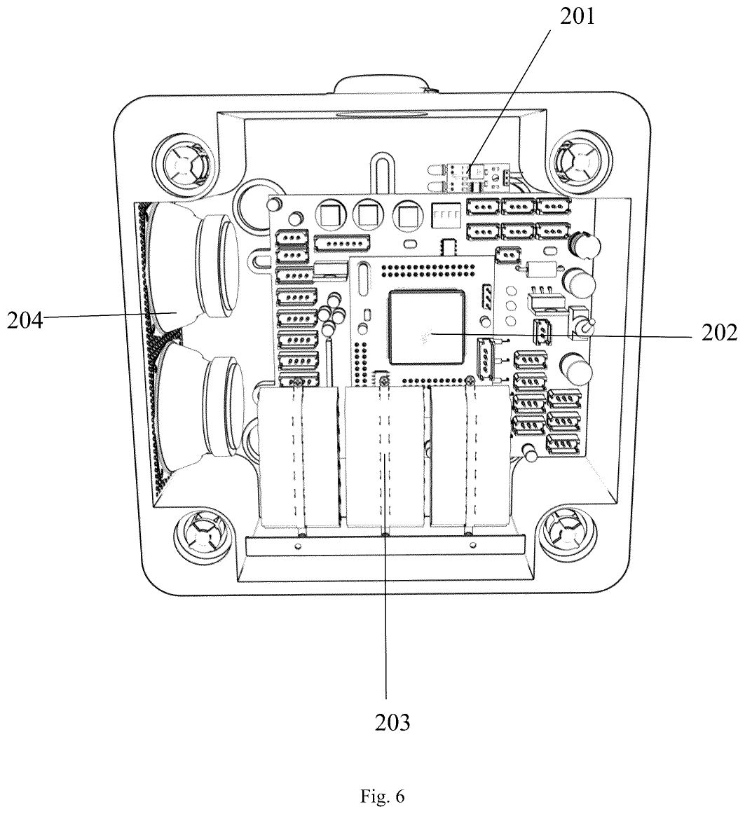

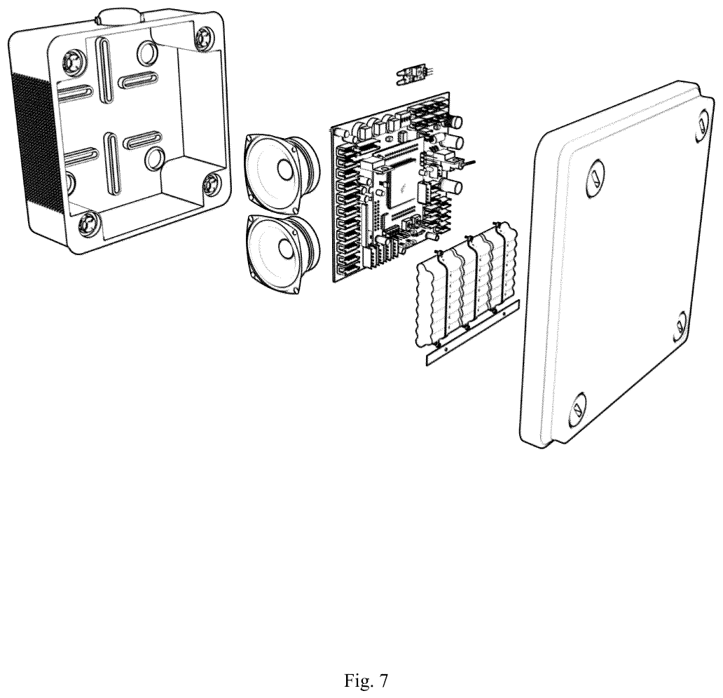

What is claimed is:

1. The invention of a device to stimulate the central nervous system of the body using electromagnetic waves to reduce the effects of multiple sclerosis and optimize the transmission of nerve messages in the axon of the nerve cell which consists of at least a wheelchair and at least one feeding unit and at least a computer system and at least wireless network and at least a magnetic field generator and at least several coils and at least 4 load cells and at least a control unit and at least a smartphone application and artificial intelligence algorithms.

2. Method and device of claim 1 in which a wheelchair with adjustable neck support is used for the equipment installation platform.

3. Method and device of claim 1 in which a generator is used to produce a pulsed magnetic field with variable parameters of frequency, intensity, field direction, time of each treatment period and time of each pulse.

4. Method and device of claim 1 in which a power supply unit is used to start the circuit of the device, where the first battery in the circuit and the supporter battery are placed in charging mode as a backup.

5. Method and device of claim 1 a computer system is used to display received information from the wheelchair control unit and send it to artificial intelligence.

6. Method and device of claim 1 in which artificial intelligence decides to change the parameters according to the parameters received from the current situation and the feedback of the patient's response to the treatment method.

7. Method and device of claim 1 in which a wireless unit is used to send and receive data between the wheelchair control unit and the computer system or smartphone.

8. Method and device of claim 1 in which at least several coils are placed along the spine and back of the patient's neck and head so that the magnetic field is induced into the nervous system through these coils.

9. Method and device of claim 1 in which the weight control unit uses 4 load cells to measure the weight and check the weight balance on the four sides of the wheelchair.

10. Method and device of claim 1 in which, if the weight is not balanced on the four sides of the wheelchair, a warning message is announced through the speaker.

11. Method and device of claim 1 in which if the weight is not balanced on the four sides of the wheelchair, it sends a signal to the mobile phone or the computer screen so that the person can correct the sitting.

12. Method and device of claim 1 in which a micro-programmed control unit is used to control the system and process information received from the computer system and artificial intelligence.

13. Method and device of claim 1 in which an artificial intelligence unit is used to adjust the treatment parameters.

14. Method and device of claim 1 in which a mobile phone application is used to enter data such as age, sex, etc. and user convenience.

15. Method and device of claim 1 in which the signal generator produces a signal in the form of sinusoidal pulses with a carrier frequency of 10 MHz, which pulses are repeated between 0.5 and 5 Hz and have a field strength between 1 and 200 milligauss.

16. Method and device of claim 1 in which the duration of the treatment is defined as once a day with duration of 12 minutes or several time intervals during the day with a shorter amount of time.

17. Method and device of claim 1 in which in order to prevent the patient from getting used to the treatment process, one of the treatment parameters in the control unit is changed every time the signal is applied.

18. Method and device of claim 1 in which the control unit records the information related to the treatment such as the frequency and amplitude of the applied electric current, the treatment time and the patient's pain level

19. Method and device of claim 1 in which by using the information received from the patient's condition, the micro only activates the embedded coil for that area that has suffered nerve damage and adjusts the parameters of the electromagnetic signal.
